(19) United States
(12) Patent Application Publication
(10) Pub. No.: US 2026/0324793 A1
(43) Pub. Date: Oct. 22, 2026

(54) Magnifying View Welding Shield Device

(71) Applicant: Todd Ritz, New Martinsville, WV (US)

(72) Inventor: Todd Ritz, New Martinsville, WV (US)

(21) Appl. No.: 19/181,521

(22) Filed: Apr. 17, 2025

(51) Int. Cl. A42B 3/20; H04N 23/69

(52) U.S. Cl. CPC A42B 3/20; H04N 23/69

(57) ABSTRACT

A magnifying view welding shield device for magnifying a view while welding includes a welding helmet having an outer shell and a viewing panel. The viewing panel has a display facing a wearer of the welding helmet. The viewing panel is operationally coupled a the processor coupled to the welding helmet. A camera is coupled to the outer shell and operationally coupled to the viewing panel through the processor to provide a visual image captured by the camera to the viewing panel. A control is coupled to the welding helmet and operationally coupled to the processor control enlargement of the image captured by the camera.

### (b) CROSS-REFERENCE TO RELATED APPLICATIONS

Not Applicable

### (c) STATEMENT REGARDING FEDERALLY SPONSORED RESEARCH OR DEVELOPMENT

Not Applicable

### (d) THE NAMES OF THE PARTIES TO A JOINT RESEARCH AGREEMENT

Not Applicable

### (e) INCORPORATION-BY-REFERENCE OF MATERIAL SUBMITTED ON A COMPACT DISC OR AS A TEXT FILE VIA THE OFFICE ELECTRONIC FILING SYSTEM.

Not Applicable

### (f) STATEMENT REGARDING PRIOR DISCLOSURES BY THE INVENTOR OR JOINT INVENTOR

Not Applicable

### (g) BACKGROUND OF THE INVENTION

### (1) Field of the Invention

The disclosure relates to welding shield devices and more particularly pertains to a new welding shield device for magnifying a view while welding. (2) Description of Related Art including information disclosed under 37 CFR 1.97 and 1.98.

The prior art relates to welding shield device. Known welding shield devices lack a controllable and adjustable magnified view while welding.

### (h) BRIEF SUMMARY OF THE INVENTION

An embodiment of the disclosure meets the needs presented above by generally comprising a welding helmet having an outer shell and a viewing panel. The viewing panel has a display facing a wearer of the welding helmet. The viewing panel is operationally coupled a the processor coupled to the welding helmet. A camera is coupled to the outer shell and operationally coupled to the viewing panel through the processor to provide a visual image captured by the camera to the viewing panel. A control is coupled to the welding helmet and operationally coupled to the processor control enlargement of the image captured by the camera.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### (i) BRIEF DESCRIPTION OF SEVERAL VIEWS OF THE DRAWING(s)

The disclosure will be better understood and objects other than those set forth above will become apparent when consideration is given to the following detailed description thereof. Such description makes reference to the annexed drawings wherein:

FIG. 1 is a top front side perspective view of a magnifying view welding shield device according to an embodiment of the disclosure.

FIG. 2 is a front view of an embodiment of the disclosure.

FIG. 3 is a rear view of an embodiment of the disclosure.

FIG. 4 is a detailed view of an embodiment of the disclosure.

FIG. 5 is a schematic view of an embodiment of the disclosure.

### (J) DETAILED DESCRIPTION OF THE INVENTION

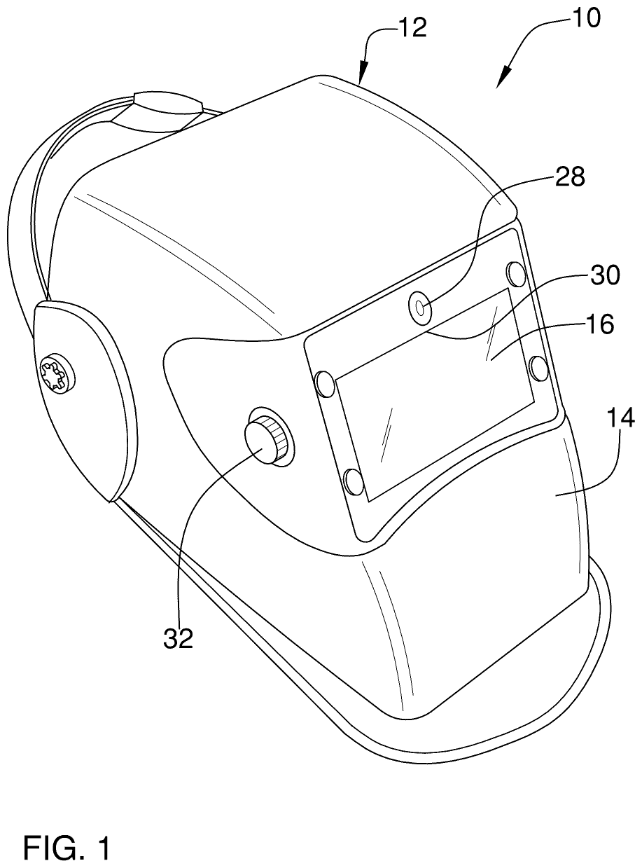

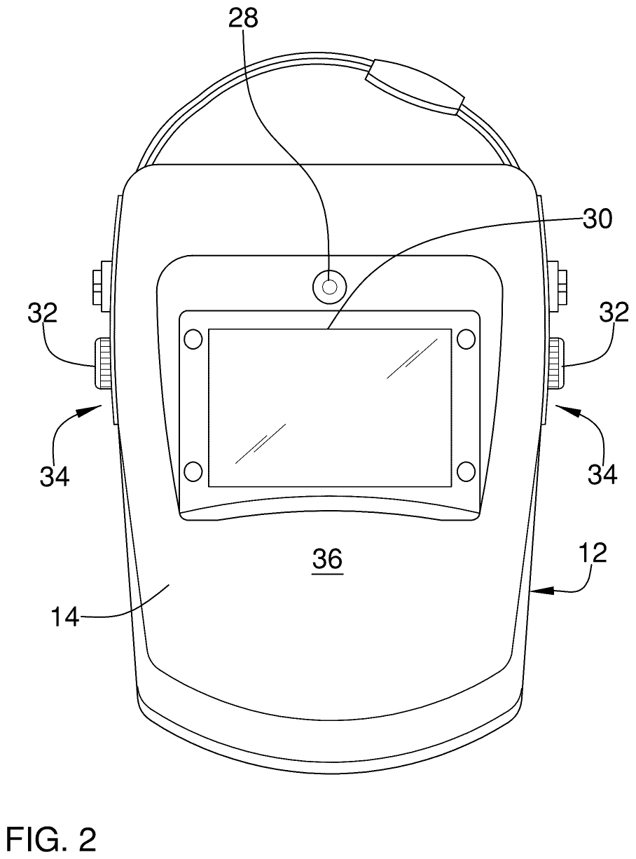

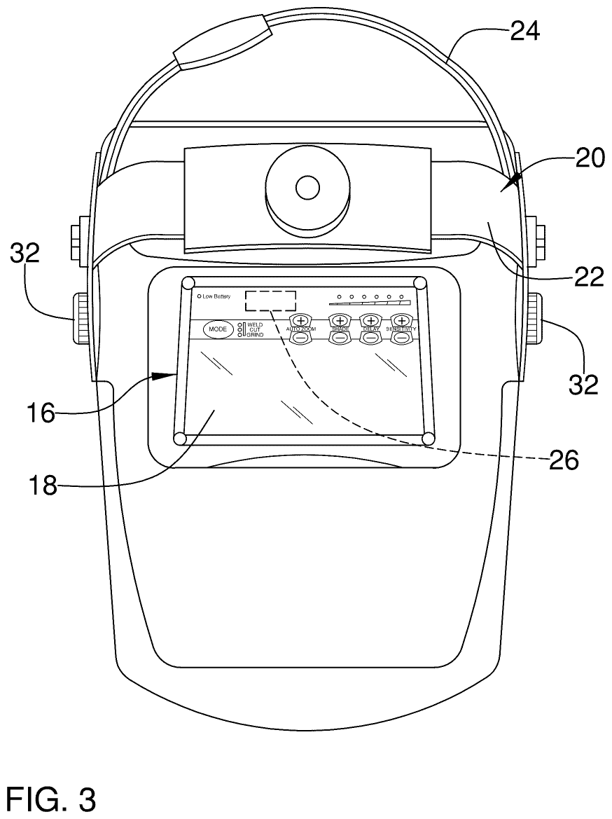

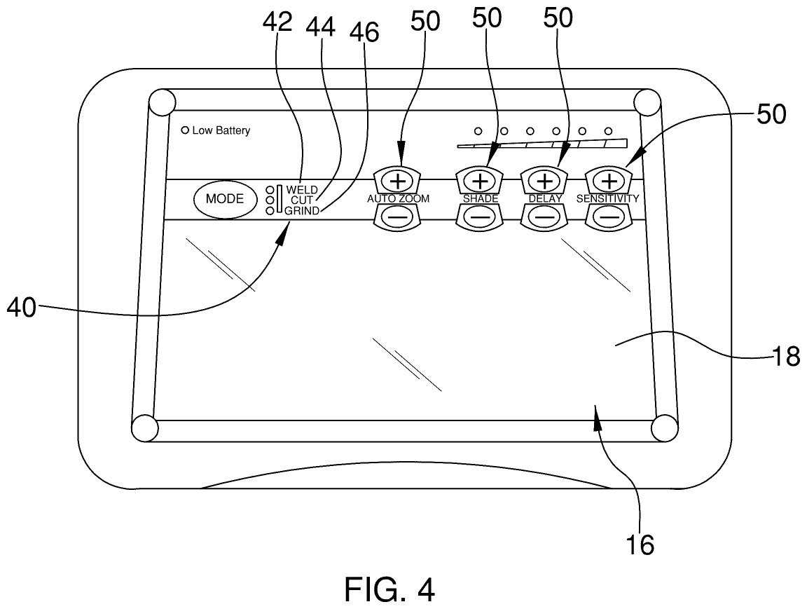

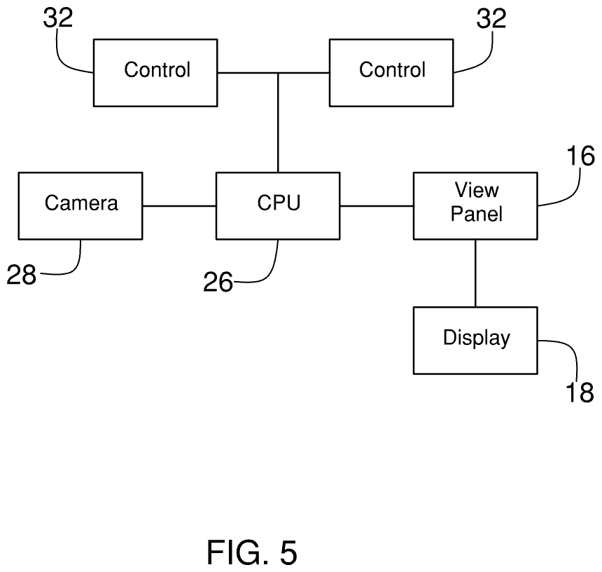

With reference now to the drawings, and in particular to FIGS. 1 through 5 thereof, a new welding shield device embodying the principles and concepts of an embodiment of the disclosure and generally designated by the reference numeral 10 will be described.

As best illustrated in FIGS. 1 through 5, the magnifying view welding shield device 10 generally comprises a welding helmet 12 having an outer shell 14 and a viewing panel 16. The viewing panel 16 having a display 18 configured to face a wearer of the welding helmet 12. The welding helmet 12 may have a harness 20 positionable on a head of the wearer including an encircling strap 22 and a top strap 24 in a conventional manner. The outer shell 14 may be pivotable relative to the harness 20. A processor 26 is coupled to the welding helmet 12. The viewing panel 16 is operationally coupled to the processor 26. A camera 28 is coupled to the outer shell 14 and operationally coupled to the viewing panel 16 by way of the processor 26. Thus, the processor 26 provides a visual image captured by the camera 28 to the viewing panel 16. The camera 28 is positioned adjacent to a top center 30 of the viewing panel 16 on the outer shell 14 of the welding helmet 12. The processor 26 is programmed for adjusting the image provided by the camera 28. The adjustment of the image through the processor 26 can be either manual, automated through pre-programming of the processor, or through manual setting of selectable automated adjustments.

Each of a pair of controls 32 is coupled to the welding helmet 12. Each control 32 is operationally coupled to the processor 26. Each control 32 is manipulatable for interaction with the processor 26 to enlarge a portion of the image captured by the camera 28. Each control 32 is positioned on a respective lateral side 34 of an outer surface 36 of the outer shell 14. Alternatively, a single control 32 may be positioned on the outer shell 14. As shown, each control 32 is a dial control allowing for rotation of the dial to scroll through menu selections, and which may be pushed or otherwise manipulated for actuation of a desired menu selection.

The processor 26 has settable modes 40 for automatically controlling a level of enlargement of the image captured by the camera 28 to provide customized enlargement parameters of the image related to selectable welding tasks. The modes 40 include a welding mode 42, a cutting mode 44, and a grinding mode 46. By selection of the desired mode 40, the processor 26 adjusts the image automatically according to desired and customized parameters related to the specific tasks being performed. The processor 26 further may have a customizable level of automated adjustment of enlargement of the image either separately or within a selected one of the modes 40 to allow for specific preferences of the user. This includes the processor 26 having a customizable level of automated adjustment of a darkening of the image, a customizable level of sensitivity to the automated adjustment of darkening of the image, and a customizable level of delay in providing the image to the viewing panel 16. Menu indicia 50 is displayed on the viewing panel 16 to allow for selection of settings for the processor 26.

In use, the welding helmet 12 is worn in a conventional manner. Either one of the controls 32 is manipulated to desired settings for the processor 26. The camera 28 captures the image and the processor 26 makes adjustments to the image to provide desired magnification. The control 32 may be further used for overriding or adjusting levels of adjustments made to the image by the processor 26.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

1. A magnifying view welding shield device comprising:
a welding helmet having an outer shell and a viewing panel, said viewing panel having a display configured to face a wearer of the welding helmet;
a processor coupled to said welding helmet, said viewing panel being operationally coupled to said processor;
a camera coupled to said outer shell, said camera being operationally coupled to said viewing panel wherein said processor provides a visual image captured by said camera to said viewing panel; and
a control coupled to said welding helmet, said control being operationally coupled to said processor wherein said control is manipulatable to enlarge a portion of the image captured by the camera for showing of the enlarged image directly on said display.

2. The magnifying view welding shield device of claim 1, further comprising said control being positioned on a lateral side of an outer surface of said outer shell.

3. The magnifying view welding shield device of claim 1, further comprising said control being one of a pair of controls, each control being positioned on a respective lateral side of an outer surface of said outer shell.

4. The magnifying view welding shield device of claim 1, further comprising said camera being positioned adjacent to a top center of said viewing panel.

5. The magnifying view welding shield device of claim 1, further comprising said processor having modes for automatically controlling a level of enlargement of said image captured by said camera wherein said processor is configured to provide customized enlargement of the image related to selectable welding tasks.

6. The magnifying view welding shield device of claim 5, wherein said modes include a welding mode, a cutting mode, and a grinding mode.

7. The magnifying view welding shield device of claim 1, further comprising said processor having a customizable level of automated adjustment of enlargement of the image.

8. The magnifying view welding shield device of claim 1, further comprising said processor having a customizable level of automated adjustment of a darkening of the image.

9. The magnifying view welding shield device of claim 8, further comprising said processor having a customizable level of sensitivity to the automated adjustment of darkening of said image.

10. The magnifying view welding shield device of claim 1, further comprising said processor having a customizable level of delay in providing the image to the viewing panel.

11. A magnifying view welding shield device comprising:
a welding helmet having an outer shell and a viewing panel, said viewing panel having a display configured to face a wearer of the welding helmet;
a processor coupled to said welding helmet, said viewing panel being operationally coupled to said processor;
a camera coupled to said outer shell, said camera being operationally coupled to said viewing panel wherein said processor provides a visual image captured by said camera to said viewing panel, said camera being positioned adjacent to a top center of said viewing panel;
a pair of controls coupled to said welding helmet, each control being operationally coupled to said processor wherein each control is manipulatable to enlarge a portion of the image captured by the camera for showing of the enlarged image directly on said display, each control being positioned on a respective lateral side of an outer surface of said outer shell; and
wherein said processor has settable modes for automatically controlling a level of enlargement of said image captured by said camera wherein said processor is configured to provide customized enlargement of the image related to selectable welding tasks, wherein said modes include a welding mode, a cutting mode, and a grinding mode, said processor having a customizable level of automated adjustment of enlargement of the image, said processor having a customizable level of automated adjustment of a darkening of the image, said processor having a customizable level of sensitivity to the automated adjustment of darkening of said image, said processor having a customizable level of delay in providing the image to the viewing panel
